(19) United States
(12) Patent Application Publication
(10) Pub. No.: US 2005/0059857 A1
(43) Pub. Date: Mar. 17, 2005

(54) Medical endoscope

(75) Inventors: Mark Freier, Oberderdingen (DE); Helmut Heckele, Knitttlingen (DE); Daniel Dietzel, Kelkheim (DE); Garrett Walsh, Houston, TX (US); William Roy Smythe, Temple, TX (US)

Correspondence Address: COHEN, PONTANI, LIEBERMAN & PAVANE, 551 FIFTH AVENUE, SUITE 1210, NEW YORK, NY, 10176, US

(73) Assignee: Richard Wolf GmbH

(21) Appl. No.: 10/877,104

(22) Filed: Jun. 25, 2004

(30) Foreign Application Priority Data
Jun. 25, 2003 (DE) 203 09 759.9

(51) Int. Cl. A61B 1/00

(52) U.S. Cl. 600/114; 600/136

(57) ABSTRACT

The medical endoscope includes a hollow shank for leading through an operating instrument and a housing which may be detachably coupled to the hollow shank. An optical system includes a shank body with a light guide and an image guide in its inside, wherein the shank body may be inserted eccentrically into the hollow shank. A plurality of hollow shanks is provided, each hollow shank having a coupling which mates with the housing and a tube having an inner wall with a diameter which is different from the inner diameter of the other tubes. The shank body with its light guide and image guide is crescent-shaped in cross section. The housing is provided with a rinsing connection union for supplying and discharging rinsing fluid through the ensoscope.

### BACKGROUND OF THE INVENTION

1. Field of the Invention

The invention relates to a medical endoscope including a housing and an optical system having an elongate shank body with a light guide and an image guide, and further including a hollow shank which can be coupled to the housing around the shank body.

2. Description of the Related Art

Such an endoscope is described in U.S. Pat. No. 4,685,449. It comprises an optical means as a first unit and a hollow shank means as a second unit, wherein both units may be detachably coupled to one another. The optics means consist of an optics housing, a shank body connected distally thereto with fiber-optics arranged therein for illuminating the intracorporal field of diagnosis or therapy, and picture guide means for observing this field, a fiber-optic connection union provided on the optics housing for connecting a fiber optic cable coming from the light source, an eyepiece union for viewing into the endoscope and a passage channel running axially through the optics housing for a medical operating instrument. The shank body with the light guide means and picture guide means and the passage channel for the operating instrument are arranged eccentrically to one another on or in the optics housing. The hollow shank means consists of the actual hollow shank and of a sub-assembly of a connection housing with rinsing connections each with a blockable supply union for the supply and discharge of rinsing fluid through the hollow shank, wherein this sub-assembly is fixedly connected to the hollow shank. This known endoscope is limited in its use inasmuch as several such endoscopes need to be available for the different anatomical conditions for the diagnosis and therapy case. These endoscopes differ from one another in that the hollow shank of the complete hollow shank means in each case have a differently sized inner diameter, in order to be able to also lead medical operating instruments with larger radial dimensions through the hollow shank means with a hollow shank which is larger in diameter. Moreover, since such endoscopes are relative expensive to manufacture on account of the hollow shank means designed in a manner which is complicated and extensive in material, the expense for the procurement of an endoscope supply with differing hollow shank means is accordingly expensive.

A further endoscope is described in U.S. Pat. No. 5,095,889. This endoscope used for laser lithotripsy has the same components as the previously described endoscope, but one difference lies in the fact that all components of this endoscope are designed as components which are fixedly connected to one another. In this case too one may only fulfill the requirements with regard to diagnosis and therapy by way of providing such endoscopes with a hollow shank which in each case has a different size of diameter.

A yet further endoscope is described in U.S. Pat. No. 6,503,196. This endoscope is essentially equal to that of U.S. Pat. No. 5,095,889, thus comprises components which are fixedly connected to one another and in its hollow shank comprises a fixedly integrated optical system with light guide means and picture guide means. This optical system occupies a semi-circular cross-sectional area of the hollow shank and is protected from the remaining cross section of the hollow shank by a diametrical separating wall. A medical operating instrument or a rinsing tube may be led through this remaining cross section. This endoscope too has the disadvantages which have been cited in context of both previously described endoscopes.

### SUMMARY OF THE INVENTION

The object of the invention lies in improving an endoscope of the initially mentioned type which by way of a construction which is simplified in its design and saves costs and material may be adapted to the respective intracorporal operations such that in the given case of operation a gentle medical operation as well as the use of a medical operating instrument with relatively large radial dimensions is possible.

According to the invention, a plurality of exchangeable hollow shanks are provided, each hollow shank having a coupling which mates to the housing, and a tube receives a shank body eccentrically, each tube having an inside diameter which is different from the inside diameter of the other tubes.

By way of this solution, one provides an endosope with a simply designed exchange shank system with a considerable space gain. This results in the significant advantage that depending of the respective case of diagnosis or therapy one may select that hollow shank with respect to its diameter which is required in order to be able to introduce the working instrument required for the respective case of diagnosis or therapy into the body cavity of the patient. The hollow shank used in each case has a sufficiently large free cross sectional lumen in order to be able to lead the respective required operating instrument through the hollow shank. While in one case a hollow shank with a relatively small inner diameter is used, in another case a hollow shank with a larger inner diameter is used. Thus one and the same endoscope according to the invention has a larger field of application since it is also possible to use operating instruments with larger radial dimensions by way of exchanging the hollow shank. The endoscope according to the invention also has significant advantages with regard the use of material and the costs. The exchange shank set saves material and may be manufactured less expensively since each hollow shank only consists of a tube part with a proximal coupling part and has no further connection and conduit parts, as is the case with the state of the art. A further advantage with regard to this lies in the fact that the rinsing connections with their conduit parts are only present once since they are integrated into the optical means as a main unit of the endoscope according to the invention. By way of the crescent-shaped optical means the intracorporal manipulation of the operating instruments whilst viewing is significantly simplified and safer, and by way of this the operation may be carried out more quickly.

One advantageous design feature of the endoscope according to the invention lies in the fact that the convex circumferential section of the crescent-shaped cross section of the shank body of the optical means borders on the inner wall surface of the respective hollow shank. By way of this design the shank body always bears essentially on the respective assembled hollow shank so that it is ensured that in each case the largest free lumen of the used hollow shank is available.

A further advantageous feature lies in the fact that the shank body additionally to its light and picture guide means comprises an instrument channel in its inside for leading through an auxiliary instrument. By way of this, further to the main operating instrument which is introduced through the free lumen of the hollow shank, it possible to also introduce an auxiliary instrument with a relatively small diameter through the endoscope to the location of diagnosis or therapy.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

FIG. 1 is a side view of an exchange shank;

FIG. 2 is a side view of the endoscope with the exchange shank removed;

FIG. 3 is a side view of the endoscope with an attached exchange shank; and

FIGS. 4, 5 and 6 are cross sections according to the section line detail in FIG. 3 with different exchange shanks.

### DETAILED DESCRIPTION OF THE PRESENTLY PREFERRED EMBODIMENTS

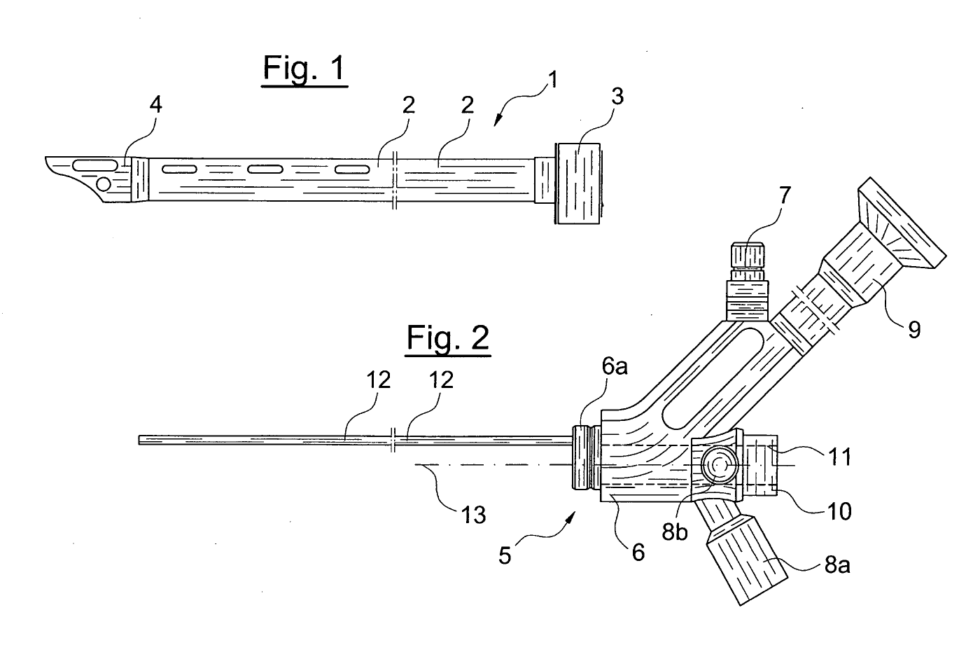

FIG. 1 shows a hollow shank 1 which consists of a tube part 2 which is preferably circular in cross section, with a proximal coupling part 3. The distal end 4 of the tube part with regard to its construction may be shaped differently, which depends on which manner and to which extent the intracorporal field of diagnosis or therapy is to be illuminated and observed. This would also depend on whether the endoscope for example is used as a bronchoscope. For the endoscope or bronchoscope according to the invention one uses two or more hollow shanks 1 in the previously described manner with the difference that each hollow shank has a different inner diameter. In the FIGS. 4, 5, and 6, for example, there are shown three hollow shanks 1 which have respectively larger inner diameters. These hollow shanks form an exchange set, and the use of the hollow shank concerned depends on which radial dimensions are used for the operating instrument used in the given case of operation and which is to be led through the hollow shank.

FIG. 2 shows an optical arrangement 5 as a main unit of the endoscope. This main unit comprises a proximal housing 6 with several connection unions. This housing then is provided with a connection union 7 for a light guide cable which is connected to an external light source, with a connection union 8a for introducing rinsing fluid, e.g. an inhalation gas on using the endoscope in particular as a bronchoscope in combination with the use for special ventilation techniques, and with a connection union 8b for leading out rinsing fluid or inhalation gas. Moreover, the housing 6 is also provided with a common eyepiece attachment as well as with a proximal connection union 9 to which for example a common introduction valve or likewise for an operating instrument (not shown) is connected.

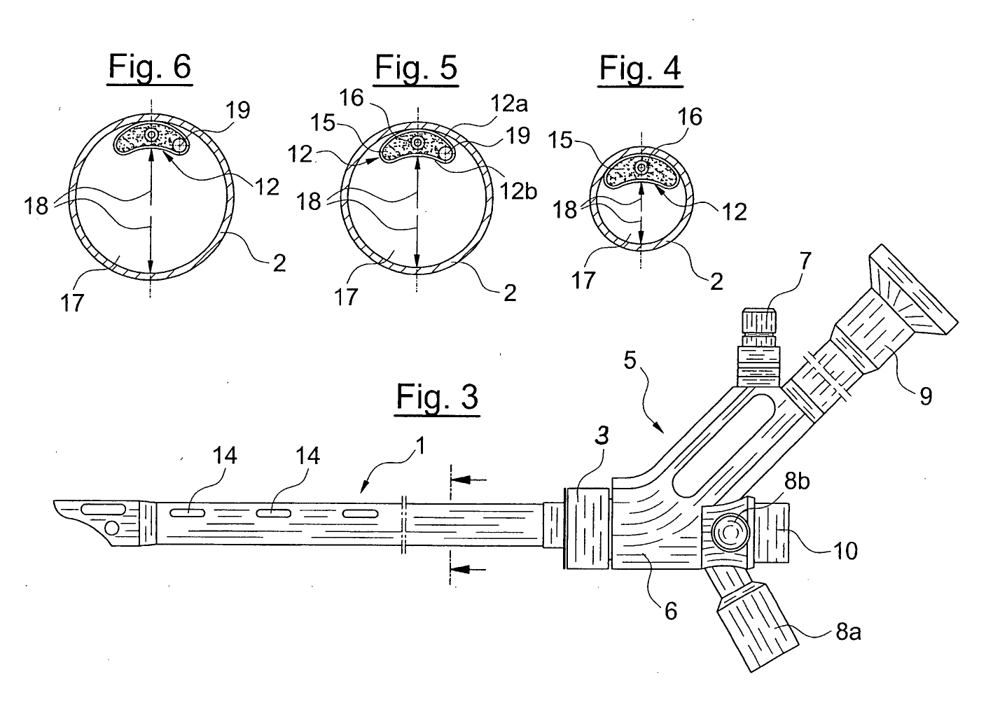

At its distal end, the housing 5 is fixedly connected to a shank body 12 in a manner such that it is eccentrically offset (FIG. 2) with respect to the longitudinal middle axis 13 of the main housing 6. The eccentric offset of the shank body 12 is such that if a hollow shank 1 is assembled on the distal coupling part 6a of the housing 6 (FIG. 3) the shank body borders on the inner wall surface of the assembled hollow shank, as is indicated at 14 in FIG. 3 and is to be recognized even better in the FIGS. 4, 5 and 6.

In a manner known per se, light guide means 15 in the form of fiber optics and image guide means 16 in the form of optical lenses and likewise, as indicated in the FIGS. 4, 5 and 6 are provided in the shank body 12 as well as in the housing 6, in order to be able to observe an intracorporal field diagnosis or therapy.

As is also evident from the FIGS. 4, 5 and 6, the shank body 12 in cross section is designed crescent-shaped and has a convex circumferential section 12a and a concave circumferential section 12b. While the convex circumferential section 12a faces the inner wall section of the respective hollow shank 1 or borders this, the concave circumferential section 12b of the shank body 12 faces the center of the hollow shank 1. Furthermore the cross section of the shank body 12 has a relatively small size area in comparison to the cross section of the hollow shank 1, as is evident from the FIGS. 4, 5 and 6. The cross section of the shank body 12 is preferably selected such that with respect to the hollow shank which is used with the smallest diameter, it is roughly only a fourth of the free lumen cross section of this hollow shank 1, as may be deduced from FIG. 4.

On account of the previously described design of the shank body 12 with the light guide and image guide means 15 and 16, in combination with the respectively used hollow shank 1 there results a relatively large free lumen 17 of the respectively used hollow shank 1 as is particularly indicated also by the arrows 18.

From the above description it is to be deduced that a hollow shank 1 according to FIG. 4 is detachably connected to the optic means 5 if an operating apparatus with relatively small radial dimensions is used. If an operating instrument is required which has larger radial dimensions, a hollow shank 1 with a larger inner diameter is detachably connected to the optical means 5, for example a hollow shank according to FIG. 5 or according to FIG. 6.

With a further design of the shank body 12 this may be additionally provided in its inside with an instrument channel 19, as is indicated in the FIGS. 5 and 6. An auxiliary instrument which is very small in diameter may be led through this additional instrument channel in order thus to become active in the intracorporal field of diagnosis and therapy. This instrument channel 19 is preferably provided in an end region of the crescent-shaped cross section of the shank body.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. An endoscope comprising:
a housing having a rinsing connection union for supplying and discharging rinsing fluid, a longitudinal axis, and a distal coupling part surrounding said axis;
an optical system comprising an elongate shank body extending through said coupling part parallel to said axis, said shank body having a crescent-shaped cross section and having therein a light guide and an image guide; and
a plurality of hollow shanks, each said hollow shank comprising a coupling which is mateable with said distal coupling part, and a tube having an inner wall with a diameter which is different from the inner diameter of each other said tube, said shank body being received eccentrically through said tube when said coupling is mated to said distal coupling part.

2. An endoscope as in claim 1 wherein said crescent-shaped cross section has a convex circumferential section which is juxtaposed against the inner wall of any one of said hollow shanks which is mated to said distal coupling part.

3. An endoscope as in claim 1 wherein said shank body further comprises an instrument channel for receiving an auxiliary instrument therethrough.
